(19) United States
(12) Patent Application Publication
(10) Pub. No.: US 2025/0121000 A1
(43) Pub. Date: Apr. 17, 2025

(54) INHALABLE BICARBONATE COMPOSITIONS AND DELIVERY SYSTEMS THEREFOR

(71) Applicant: Nebulize Performance Inc., Grand Valley (CA)

(72) Inventor: Christopher Geoffrey Rudolf Perry, Acton (CA)

(21) Appl. No.: 18/915,188

(22) Filed: Oct. 14, 2024

Related U.S. Application Data
(60) Provisional application No. 63/590,194, filed on Oct. 13, 2023.

(51) Int. Cl. A61K 33/00; A61K 9/00; A61M 16/06; A61M 16/20; A61P 21/00

(52) U.S. Cl. CPC A61K 33/00; A61K 9/0078; A61M 16/06; A61M 16/208; A61P 21/00

(57) ABSTRACT

Provided are inhalable bicarbonate compositions for treating acidosis and enhancing athletic performance, training and recovery. The compositions include 0.5% to 5%, and preferably about 3.5% bicarbonate, by weight, dissolved in water. Methods are provided for treating acidosis and enhancing athletic performance. The method includes inhaling an effective amount of an inhalable bicarbonate composition, via a delivery system, prior to engaging in physical activity and/or between bouts of physical activity and/or following physical activity. Also provided are delivery systems for inhalable bicarbonate. The delivery systems include an adjustable variable speed pump for optimal and rapid delivery of bicarbonate according to the user's preference and comfort.

### TECHNICAL FIELD

The embodiments disclosed herein relate to administering bicarbonate for acidosis, and, in particular to inhalable bicarbonate compositions and delivery systems therefor.

### INTRODUCTION

Metabolic acidosis occurs when there is excess acid in the body, which can be due to the overproduction of acids by high intensity exercise, metabolic processes, the inability of the kidney to remove excess acids from the body, and/or the loss of too much sodium bicarbonate from the body. Metabolic acidosis is a broad condition and can be classified into several subtypes, including diabetic acidosis, hyperchloremic acidosis, kidney disease, and lactic acidosis. Respiratory acidosis occurs when carbon dioxide accumulates in the blood or tissues due to reduced pulmonary ventilation or pulmonary gas exchange thereby creating carbonic acid. Acidosis can also occur when an individual is exposed to gases that create acids once dissolved in the water component of the body, such as chlorine gas.

In addition, subtypes of acidosis can be present in a number of clinical and non-clinical conditions including cancer, liver failure, hypoglycemia, and exercise of high intensity or long duration. Currently, the treatment of acidosis primarily involves systemic administration of sodium bicarbonate via intravenous or oral means. While these methods can be effective, they have several drawbacks including, slow onset of action, frequent dosing, and potentially debilitating side effects that often limit its use.

Specific to exercise, metabolic acidosis is a major contributor to muscle fatigue, limiting endurance, strength, and overall athletic performance. Athletes and fitness enthusiasts share a common interest in identifying new strategies to improve exercise performance, whether that be during an event or competition itself, or through mechanisms to enhance training and accelerate recovery. Within the context of high intensity exercise, a faster speed is essential to winning sprint-style competitions across a broad array of sports such as track and field and soccer among others. Athletes in these events commit themselves to years of structured exercise training to achieve their potential for maximum speed and muscular endurance. However, the potential of most athletes are remarkably similar. Indeed, the difference in maximum speed between the top contenders in an event is often a mere fraction apart from each other. As such, any additional strategy to boost speed and endurance beyond the results achieved with conventional exercise training could make the difference between winning or losing a competition. For this reason, athletes are increasingly motivated to seek additional strategies to enhance the improvements in speed that are achieved with exercise training.

In order to develop such ‘ergogenic’ enhancements of speed, an examination of the physiological limitations of exercise is required. High-intensity or sprint-type exercise cause rapid muscle fatigue, which limits maximum speed as well as endurance, i.e., the length of time that a given speed can be sustained. The causes of this fatigue are multi-factorial, but a large body of evidence suggests an accumulation of metabolic acids, i.e., acidosis is an important contributor (Robergs et al., Int J Sport Nutr Exerc Metab, 15 (1): 59-74, 2005). Metabolic acids are natural by-products of energy metabolism within muscles (Robergs et al., Am J Physiol Regul Integr Comp Physiol. 287 (3): R502-516, 2004). However, they interfere with a variety of specialized enzymes that are critical for muscle contraction (Robergs et al., 2004; Robergs et al., 2005). Indeed, a lower pH (acidic environment) is seen in muscle with repeated short-sprints, which place enormous demands on a multitude of enzymatic pathways (Robergs et al., 2004). It follows that the greatest drop in pH/accumulation of acids is seen when the athlete can no longer run, which likely corresponds with acid-interference of muscle contraction.

This lower pH is also seen in the blood because of a ‘spill-over’ of acids from the muscle into the bloodstream. Thus, measuring blood pH is used as an index of acid accumulation in muscle, which has allowed coaches, athletes and scientists alike a simple approach to monitoring such acidosis during exercise via small finger-prick blood sampling, thereby eliminating the need for invasive muscle biopsy sampling. With this approach, a variety of studies have demonstrated that pre-exercise oral consumption of sodium bicarbonate (i.e., ‘baking soda’) delays the decline in blood pH associated with acidosis, maintains higher alkalized blood concentrations pre-exercise, during, and post-exercise compared to those without the bicarbonate supplement and improves sprint performance (increase endurance) (Raymer et al., J Appl Physiol. 96 (6): 2050-2056, 2004; Mero et al., J. Strength Cond. Res. 18:306-310, 2004; Ducker et al., J Strength Cond Res. 27 (12): 3450-3460, 2013; Zajac et al., J Sports Sci Med. 8 (1): 45-50, 2009) due to the alkaline properties of this compound.

The use of sodium bicarbonate as an ergogenic aid is feasible given this compound naturally occurs in both blood and muscle (Robergs et al., 2004). This compound is the major acid-buffering mechanism in these compartments and serves to maintain a normal and healthy blood pH throughout the day (Robergs et al., 2004). However, natural concentrations of sodium bicarbonate are not capable of preventing acidosis during exercise. Thus, the ability of sodium bicarbonate to increase endurance during fast sprints has been associated with a slower rate of blood acidosis as seen by a delayed decline in pH during exercise. Such improvements were typically seen as <5% faster sprint times, but this is considerable given the difference in time between athletes in top-ranked competitions can often be less than 1-2%.

While these studies have shown great promise of ingesting sodium bicarbonate to improve sprint performance, the procedure is not commonly practiced by athletes given considerable negative side effects (Carr et al., International Journal of Sport Nutrition and Exercise Metabolism, 21:189-194, 2011). Large amounts of sodium bicarbonate are required to improve running performance (upwards of 20 grams) given 1) the stomach contains naturally occurring digestive acids that must first be neutralized; and 2) the liver acts as a filter to limit the amount of bicarbonate that enters the circulation. The gastric side effects could potentially impede exercise performance and actually render consumption of sodium bicarbonate as counter-productive. In this light, the utility of sodium bicarbonate to improve sprint exercise is limited unless a strategy can be developed to bypass the barrier of stomach acid and the potential role of the liver's filtration mechanisms.

Given that sodium bicarbonate naturally occurs in the blood, in water and in many foods, it is not listed on the World Anti-Doping Agency list of banned supplements for athletes (accessible at <https://www.wada-ama.org/sites/default/files/2023-05/2023list_en_final_9_september_2022.pdf>). Indeed, oral ingestion is performed by some athletes without penalty or violation of accepted procedures, but the lack of popularity in oral ingestion of sodium bicarbonate is due to the digestive side effects.

Accordingly, there is a need for novel methods of administering bicarbonate for acidosis that overcome the difficulties of orally ingested sodium bicarbonate and are in compliance with accepted and ethical procedures for sports training and clinical treatment.

### SUMMARY

The present disclosure provides a novel approach to treat acidosis by inhalation of bicarbonate (e.g., sodium bicarbonate). Inhalation of bicarbonate addresses the limitations of current methods of bicarbonate administration and has the potential to benefit millions of people suffering from clinical or non-clinical acidosis. While the following description pertains to the treatment of metabolic acidosis during exercise specifically, the embodiments disclosed herein may be applied to any medical and non-medical conditions where acidosis may occur, irrespective of the underlying cause.

The present disclosure relates to the inhalation of sodium bicarbonate—which can be used to treat acidosis—as well as device(s) and pre-mixed sodium bicarbonate formulations for the delivery thereof.

The present disclosure demonstrates that inhalation of low-dose nebulized bicarbonate can significantly improve exercise performance in a repeated-sprint test that models athletic protocols from diverse sports. In a preferred embodiment, the form of bicarbonate is nebulized/aerosolized; however, other forms are also contemplated, e.g., vaporized, atomized. Furthermore, nebulized bicarbonate exhibits a constant effect in improving performance from early sprints to late sprints of each set in a repeated-sprint test. Nebulized bicarbonate was shown to be an effective and practical method of intervention without detrimental side effects, as none were observed or reported.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a diagram of a sprint exercise protocol;

FIG. 2 is a chart showing average sprint times for inhaled bicarbonate and placebo conditions over 4 sprint sets;

FIG. 3 is a chart showing average sprint times for inhaled bicarbonate and placebo conditions in early-phase sprints within each set shown in FIG. 2;

FIG. 4 is a chart showing average sprint times for inhaled bicarbonate and placebo conditions in late-phase sprints within each set shown in FIG. 2;

FIG. 5 is a diagram of a delivery system, according to an embodiment;

FIG. 6 is a flow chart of a method for enhancing athletic performance, according to an embodiment; and

FIG. 7 is a face-worn delivery system, according to an embodiment.

### DETAILED DESCRIPTION

Various apparatuses, compositions or processes will be described below to provide an example of each claimed embodiment. No embodiment described below limits any claimed embodiment and any claimed embodiment may cover processes, apparatuses or compositions that differ from those described below. The claimed embodiments are not limited to compositions, apparatuses or processes having all of the features of any one composition, apparatus or process described below or to features common to multiple or all of the embodiments described below.

The term “about,” or a tilde (˜) when preceding a value herein, refers to the usual error range for the respective value readily known to the skilled person in this technical field. For example, for bicarbonate percent by weight±0.1%, are within the intended meaning of the recited value “about 1%” or “˜1%”; for bicarbonate solution by volume±0.1 mL, are within the intended meaning of the recited value “about 1 mL” or “˜1 mL”.

The term “acidosis,” as used herein, refers to all forms of clinical or non-clinical acidosis including diabetic acidosis, hyperchloremic acidosis, lactic acidosis, metabolic acidosis and respiratory acidosis, regardless of the underlying disease, condition or cause, including exposure to agents that acidify the blood. Although certain embodiments herein are described in relation to a sub-type of metabolic acidosis, lactic acidosis, caused by performing high intensity or long duration exercise, it should be understood that those embodiments are applicable to other forms of acidosis.

The term “inhalable,” as used herein, means in a form capable of being atomized, nebulized, vaporized or aerosolized for inhalation into the lungs and absorption into the bloodstream.

Inhalable bicarbonate compositions for treating or delaying the onset of acidosis are described herein. In preferred embodiments, the form of bicarbonate is sodium bicarbonate, however, other forms or salts of bicarbonate (adjusted for percent weight) are also contemplated, e.g., potassium bicarbonate. According to some embodiments, potassium bicarbonate is substituted for sodium bicarbonate. According to other embodiments, mixtures of sodium bicarbonate with potassium bicarbonate, or other bicarbonate salts are contemplated.

The inhalable bicarbonate compositions were prepared by mixing (e.g., by agitation) an effective amount of sodium bicarbonate in water. In different embodiments, the bicarbonate composition includes sodium bicarbonate of about 0.5%, about 1%, about 1.5%, about 2%, about 2.5%, about 3%, about 3.5%, about 4%, about 4.5%, or about 5% w/w dissolved in water. In a preferred embodiment, the amount is about 3.5% w/w of sodium bicarbonate dissolved in water. This amount of bicarbonate is approximately 95% less than in previous studies where sodium bicarbonate was administered orally i.e., ingested (Grgic, J. et al. J. Int. Soc. Sports Nutr., 2021, 18:61; Grgic, J. et al. J. Int. Soc. Sports Nutr., 2021, 18:71).

According to some embodiments, the inhalable bicarbonate composition may further include flavoring e.g., fruit extract, vanilla, menthol, etc. According to some embodiments, the inhalable bicarbonate composition may include solubilizing, aerosolizing or stabilizing agents such as polyethylene glycol, glycerin, etc.

According to some embodiments, the inhalable bicarbonate composition may include nanoparticles to enhance the aerosolization of inhalable bicarbonate to optimize efficiency of bicarbonate delivery. The nanoparticles may improve the dispersion of sodium bicarbonate particles, enabling better absorption and bioavailability.

The inhalable bicarbonate compositions described herein may be provided pre-mixed in screw cap vials, disposable cartridges, squeeze bottles, dropper bottles, and the like. According to some embodiments, the bicarbonate composition may be provided in powder form in pouches or sachets for mixing with an appropriate amount of solvent (e.g., water).

According to some embodiments, the bicarbonate composition may be provided as dissolvable inhalation strips infused with sodium bicarbonate. The strips may be placed directly on the tongue or inside the mouth, where they quickly dissolve, releasing the bicarbonate for inhalation and absorption through the respiratory system.

### Materials & Methods

### Research Participants

Participants were recruited on the basis of being sub-elite athletic males aged 21 to 26 years old, having played soccer at high levels of competition, specifically semi-pro and competitive ‘rep’ (representative) teams, and in positions that involve repeated running sprints (i.e., defenders, midfielders and strikers). The participants had an average Body Mass Index of 22.7±1 kg/m³ and engaged in training or competition 4 times per week on average. None of the participants were smokers or regular users of tobacco products or reported heart disease, rheumatoid arthritis, diabetes, poor lung function, uncontrolled high blood pressure (hypertension), or any other major health disorder.

### Experimental Design

A randomized, single-blinded, crossover design was used. Participants completed a repeated sprint exercise protocol to total exhaustion in an indoor facility after inhaling 20 mL of a 3.5% sodium bicarbonate solution (700 mg) dissolved in water or a placebo (pure water) prior to the sprint test. Participants inhaled the solutions through a commercially available nebulizer (Pari Vios Nebulizer System™) for 30 min while seated at rest over 5 bouts of 6 min (4 mL/bout) prior to completing the first sprint set and were required to breathe the same solution for 60 seconds in between each set (described below).

The order of placebo or bicarbonate was assigned with a numbering system of 1 and 2 respectively, where half of the participants were randomly assigned a 1-2 vs 2-1 order with each trial separated by one week. Following the last trial, participants were asked to guess the order of trials. 50% guessed incorrectly with the other 50% indicating the correct order. Participants were also asked to describe any noticeably breathing difficulties including chest tightness, shortness of breath or any sign of respiratory difficulty, as well as nausea. All participants indicated that they were free of any such distress.

The study was conducted during the month of November where participants finished their outdoor soccer season and were in the initial stages of their indoor season. Participants were instructed to maintain their regular routines/schedules and not to alter their diet or training regimen during the study. All test trials were scheduled prior to any intense training/practices participants may have had with their affiliated teams.

### Repeated Sprint Exercise Test (X2)

After inhaling a nebulized bicarbonate solution for 30 min, an 8 min break was given before starting a sprint exercise protocol. FIG. 1 is a diagram of the sprint exercise protocol. A sprint set involved 6×40 m shuttle sprints with a 25 second rest between each shuttle sprint. A shuttle sprint involves sprinting 20 m from a starting point to a pivot point in a straight line, turning at a pivot point and immediately running a 20 m return sprint back to the starting point. A 2 min rest was provided between sprint sets at which time participants were given 30 seconds to rest before inhaling the same nebulized solution for 1 minute, followed by a final 30-second break breathing room air. The sprinting exercise protocol required participants to complete as many sprint sets (i.e., 6×40 m sprints with the intervening breaks) as possible until voluntary total exhaustion (TE) was achieved, and no further sprints could be completed.

Sprint times were measured by Brower Training System™ laser timing gates. The sprint exercise protocol was repeated in the placebo and bicarbonate trials I separated by 7 days. All participants completed at least 4 sprint sets, with most of the participants completing 5 and 6 sprint sets. However only sprint sets 1-4 were statistically analyzed based on a complete sample size.

### Statistical Analysis

Data was expressed as time (in seconds)+SEM. A two-way repeated measures ANOVA was executed to assess the difference in average sprint times for the bicarbonate and placebo trials by comparing average time for sprints 1 to 3, 4 to 6, and overall set times (sprints 1 to 6) for all sets, as well as the effect of repeated sprints over time on running time to verify the protocol was effective at creating fatigue.

### Results

### Overall Sprint Time: Average of Sprints 1-6 in Each Set

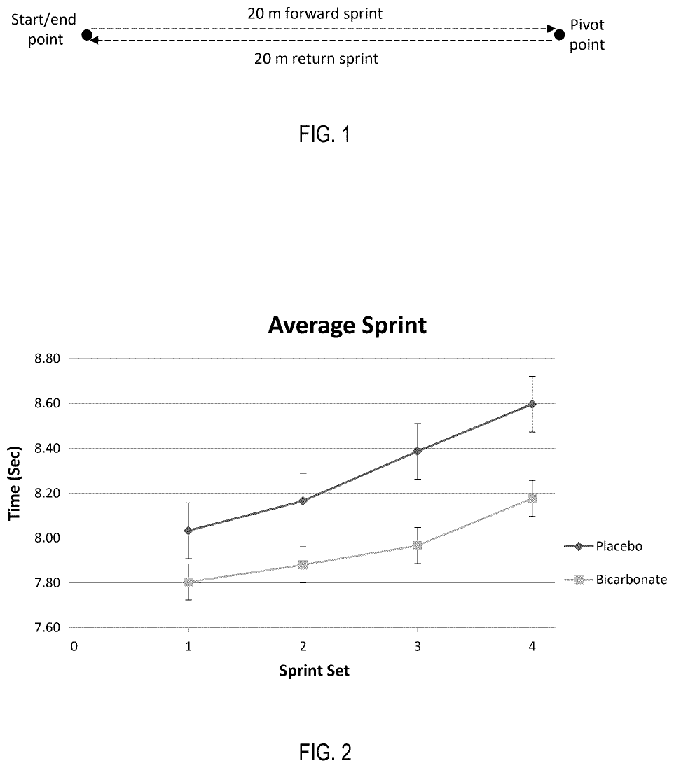

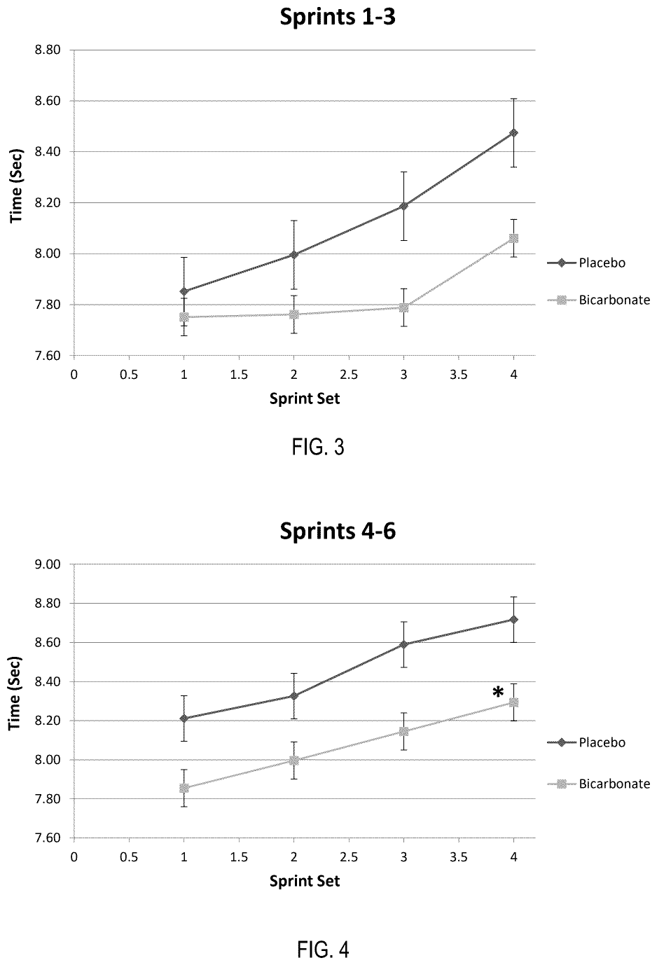

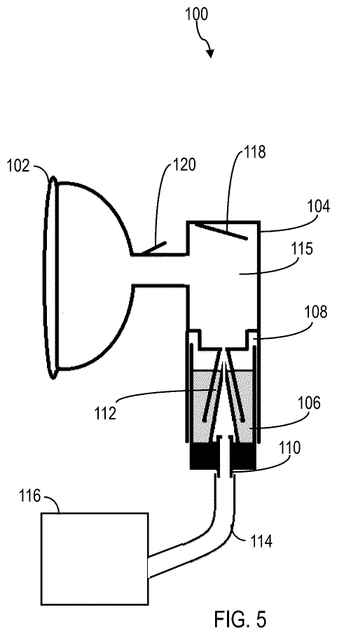

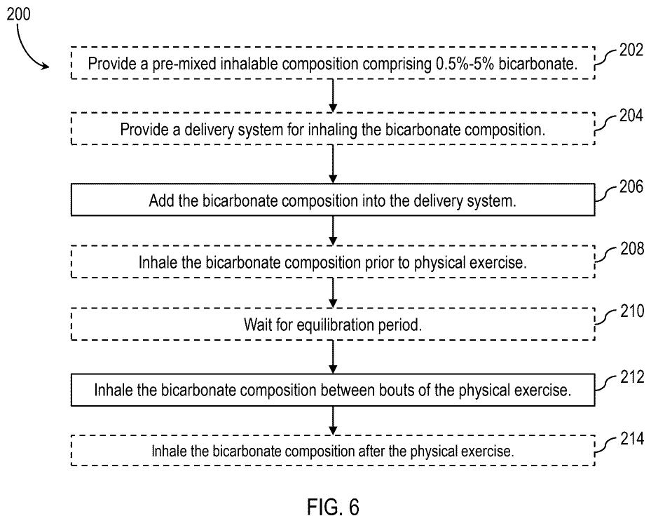

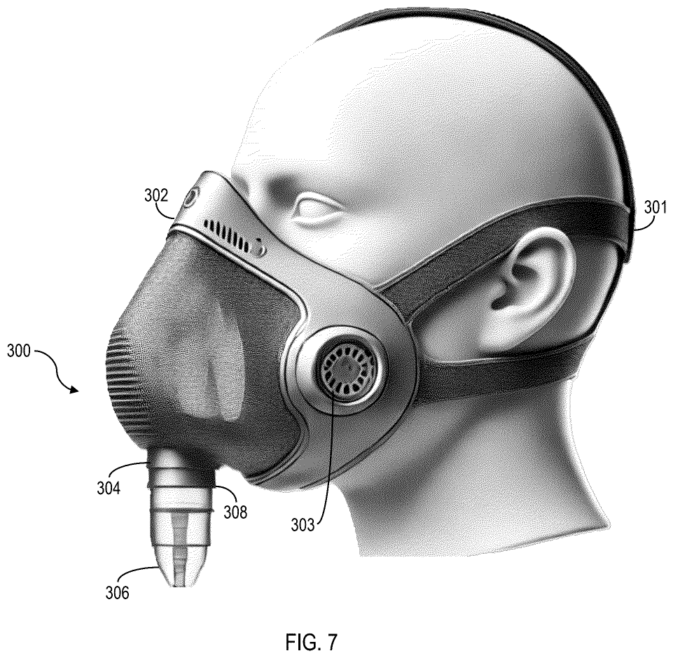

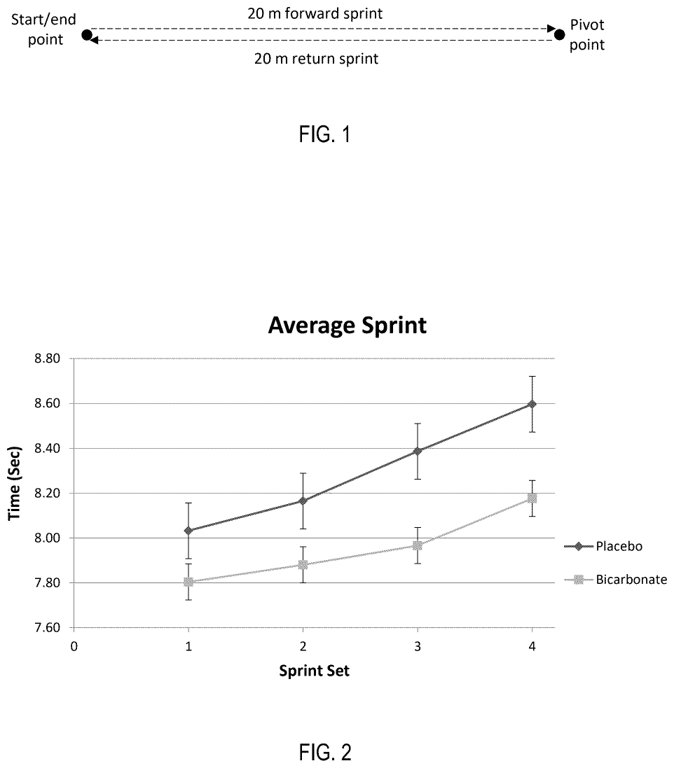

FIG. 2 shows average sprint times per set in each of the inhaled bicarbonate and placebo conditions. A main effect of time was observed whereby sprint time tended to increase for later sets in both conditions (p<0.05), thereby confirming the fatiguing nature of the sprint protocol. Bicarbonate inhalation significantly decreased sprint time by 4.1% vs placebo (p<0.05, main effect of treatment) indicating a faster running speed during highly fatiguing exercise.

### Early and Late Phase Responses: Average Time of Sprints 1-3 vs Sprints 4-6 in Each Set

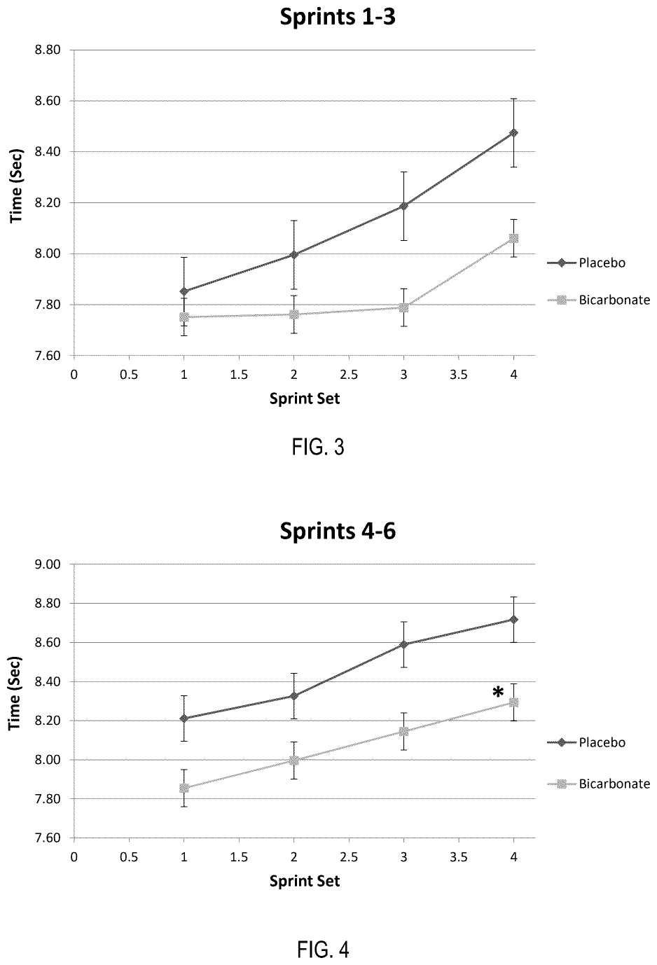

The beneficial effects of bicarbonate were also observed in the first half of each set (i.e., shuttle sprints 1-3, FIG. 3) with an average improvement in sprint time of 3.5% (p<0.05, main effect of treatment). A similar improvement of 4.5% was seen in average time during sprints 4-6 of each set (FIG. 4, p<0.05, main effect of treatment). Of the participants that completed more than 4 sprint sets, a non-significant trend was noted whereby bicarbonate tended to improve performance by 5% in sprint set 5 and 7% in sprint set 6.

### DISCUSSION

This is the first study to examine the effects of nebulized sodium bicarbonate in a repeated-sprint test that simulated an exercise regimen commonly performed within competition of various sports. Inhaled sodium bicarbonate demonstrated an overall 4.1% improvement across 4 sets of 6×40 m sprints as compared to inhaling a placebo (water). These observations agree with the hypothesis that low-dose sodium bicarbonate supplementation inhaled through a nebulizer would delay fatigue and improve running time during high-intensity sprints.

Improvement in exercise performance following oral ingestion of bicarbonate has been shown in several studies including up to ˜5% faster times in those studies that utilized a repeated-sprint test (Bishop et al., Med. Sd. Sports Exerc., 36:807-813, 2004; Price et al., Med. Sci. Sports Exerc., 35:1303-1308, 2003; Bishop et al., Med. Sci. Sports Exerc., 37 (5): 759-67, 2005; Lavender et al., Br J Sports Med, 23:41-45, 1989). These studies often employed a supplementation protocol requiring ingestion of over 20 grams of sodium bicarbonate. The present study showed a similar 4.1% significant improvement on overall sprint times over 4 sets but achieved this by inhaling significantly less bicarbonate totaling less than 1 gram. This dose represents a substantial reduction in effective bicarbonate concentrations, which suggests the lungs are less of a barrier to bicarbonate absorption into the blood as compared to the stomach and liver following oral ingestion.

Interestingly, the results obtained with nebulized bicarbonate contradicted previously published literature. In the studies by Ducker et al., 2013 and Bishop et al., 2004 it was demonstrated that bicarbonate supplementation only had an effect on improving performance in the late phase of final sprint sets. The significant improvement found in early phase sprints 1-3 (FIG. 3) and late-phase sprints 4-6 (FIG. 4) as well as overall average time of each set (FIG. 2) indicates that nebulized bicarbonate has a constant and consistent effect in improving performance across early to late sprints. This finding suggests that nebulized bicarbonate has a more immediate and sustained impact on performance compared to a delayed effect with ingested bicarbonate despite the large amounts required for ingestion.

All participants completed 4 sprint sets, with most of the participants completing 5 and 6 sprint sets. However only sprint sets 1-4 were statistically analyzed due to the requirement for a full sample size. However, in participants that completed more than 4 sprint sets, the trend with improved performance continued with a non-significant 5% faster time in sprint set 5 and 7% in set 6. It is possible that nebulized bicarbonate's positive effects seen in sets 1-4 are sustained or greater in later sets but this would only benefit those participants with a sufficiently high fitness level to achieve additional sprint sets. Nevertheless, the utility of inhaling nebulized bicarbonate is robust with clear benefits to prolonged sprint type exercises, consistent with previous findings that bicarbonate ingestion benefits sprint activities (Mero et al., 2004; Bishop et al., 2004; Mueller et al., J Int Soc Sports Nutr. 10 (1): 16, 2013; Ducker et al., 2013; Bishop et al., 2005).

It has been commonly reported that although ingested bicarbonate improves performance, the common deterring side effect is gastrointestinal distress with abdominal cramps and diarrhea as examples (Carr et al., 2011). Conversely, in this study no participants experienced any symptoms of gastrointestinal discomfort or side effects with inhaled bicarbonate. This is likely because the bicarbonate does not pass through the GI tract as it does with ingestion, and is potentially able to diffuse directly into the blood via the alveoli of the lungs. The dose was clearly low enough to avoid any self-reported respiratory discomfort or breathing problems, which further supports the utility of inhaling low-dose nebulized bicarbonate as a novel ergogenic aid for improving sprint performance that is advantageous over traditional high-dose oral ingestion approaches.

After participants completed both trials, they were asked to describe their experience without knowing the order they performed their trials in. Through their responses, it was shown that half of the participants guessed the order of trials incorrectly vs half guessing correctly. This suggests there was no appreciable cue to the participants such as distinct taste or sensation associated with inhaling relatively low amounts of sodium bicarbonate compared with water. Furthermore, all participants indicated they were free of respiratory distress and did not self-report any impairment in respiratory function during both trials. This suggests that nebulized bicarbonate is a safe and practical application with no obvious side effects of discomfort or impediment to exercise.

### Delivery System

Standard commercial nebulizers comprise a pump that forces liquid through a plastic membrane containing microscopic pores that separate the liquid into a fine mist that travels along a hose to a mouth or nosepiece used to inhale the aerosol mixture. Typically, the pump is contained within a small box that is several inches in each dimension and sits on a tabletop. The box contains a small vial that holds several milliliters (mL) of water. The disadvantage of these systems for sports applications are the time-consuming labor involved in pre-mixing solutions and the single pump speed that is not adaptable to individuals of varying resting ventilation rates. For example, during the trials described above, use of a commercially available nebulizer made it challenging for the athlete to inhale all the nebulized solution with comfort due to the difference between the rate of breathing and the rate of the nebulizer pump.

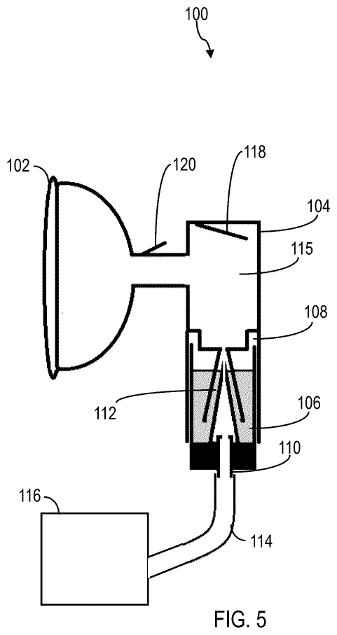

Referring to FIG. 5, shown therein is a diagram of a delivery system 100 for inhalable bicarbonate compositions, according to an embodiment. According to various embodiments, the delivery system may be a nebulizer, a vaporizer, an atomizer, or the like, for generating inhalable bicarbonate. It should be noted that the elements in FIG. 5 are not drawn to scale and are for illustrative purposes only.

The delivery system 100 includes a facemask 102 for covering both the nose and the mouth of the user. The facemask 100 is generally shaped to match the contours of a human face and is preferably constructed of a pliable material (e.g., rubber or plastic) to substantially form a seal around the user's nose and mouth when the facemask 100 is pressed to the user's face. The facemask 100 may include a strap (not shown) for wrapping around the user's head to secure the facemask 100 over the user's nose and mouth.

According to other embodiments, the face mask 100 may comprise, or be substituted with, a nasal cannula for targeted inhalation, a mouthpiece for more precise dosage control, or a specialized aerosol hood. Such embodiments may be employed according to user preferences and comfort to optimize delivery for individuals of all ages and needs.

The delivery system 100 includes a housing 104 and a reservoir 106. The reservoir 106 is for containing an inhalable bicarbonate composition and is removable from the housing 104 to add the inhalable bicarbonate composition thereto. The reservoir 106 includes a top shaped to form fit or interference fit (“snap-seal”) with a receptacle 108 in the housing 104. The housing 104 may include a tab, a button, or the like, that when depressed, disengages the snap-seal to release the reservoir 106 from the receptacle 108. According to some embodiments, the reservoir 106 is a quick load cartridge containing a premixed amount of inhalable bicarbonate composition.

The reservoir 106 includes a bottom having a connector 110. A first end of the connector 110 is in fluidic connection with an inlet of an aerosolizing mechanism 112 disposed within the reservoir 106. The aerosolizing mechanism 112 is configured to generate inhalable bicarbonate particles when a bicarbonate solution is passed through the aerosolizing mechanism 112 at pressure. A second end of the connector 110 is in fluidic connection with a gas delivery hose 114 from a variable speed pump 116. The gas delivery hose 114 is of sufficient width to allow for user-selected higher or lower speeds of the pump 116.

During operation of the delivery system 100, the facemask 102 is placed over the user's mouth and nose and the pump 116 is turned on. The pump 116 forces compressed air through the aerosolizing mechanism 112 inlet via the delivery hose 114. The compressed air forces the bicarbonate solution in the reservoir 106 through an outlet of the aerosolizing mechanism 112 at pressure thereby generating inhalable bicarbonate particles. The inhalable bicarbonate particles pass into an air chamber 115 of the housing 104 where air is mixed with the inhalable bicarbonate.

When the user inhales, a one-way inspiratory valve 118 opens to allow external air into the air chamber 115 to mix with the inhalable bicarbonate particles. The inhalable bicarbonate particles/air mixture then passes into the facemask 102 and is inhaled through the user's nose and/or mouth. When the user exhales, a one-way expiratory valve 120 between the air chamber 115 and the mouthpiece 102 seals to prevent backflow of exhaled air into the air chamber 115 to minimize inhalable bicarbonate loss. Together, the air chamber 115 and valves 118, 120 form a conduit for passing the inhalable bicarbonate from the aerosolizing mechanism 112 outlet to the facemask 102.

According to some embodiments, the delivery system may be configured to activate only when the patient inhales, ensuring efficient drug delivery and minimizing wastage. This could lead to improved patient compliance and more economical use.

The variable speed pump 116 may be adjusted by the user to reduce the amount of time required to inhale, for example, 20 mL of inhalable bicarbonate solution and ensure optimal and rapid delivery of the mixture to the athlete prior to a training or competitive event. Also, as the user's breathing rate changes during physical activity, the pump 116 may be adjusted to parallel the change in the user's breathing.

According to some embodiments, the delivery system 100 including the variable speed pump 116 is sized to be portable and easily carried by the user. For example, a compact, handheld inhalation device, similar to an inhaler/puffer, that utilizes microfluidic technology to convert sodium bicarbonate solution into an ultra-fine mist. The handheld inhalation device may incorporate a miniature pump, a power source (a battery), and a disposable cartridge or reservoir containing the sodium bicarbonate composition. This would allow users to carry the device with them and administer treatment on the go.

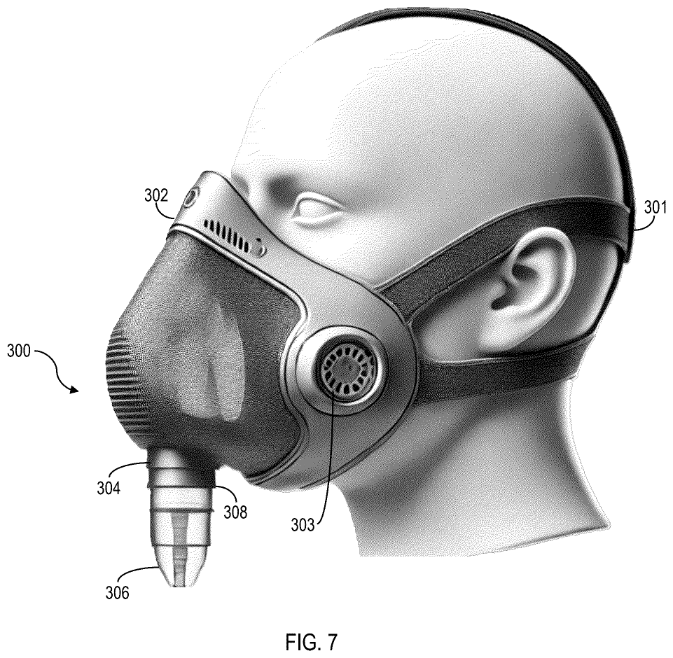

According to another embodiment shown in FIG. 7, there is a face-worn delivery system 300. The delivery system 300 is substantially similar to the delivery system 100, having a housing 304 integrated into a facemask 302. The facemask 302 covers at least the mouth, or the mouth and the nose, of the user and includes strapping 301 for securing the delivery system 300 to a user's face. The face mask 302 includes inhalation and/or exhalation ports 303.

The housing 304 includes a pump, an aerosolizing mechanism, a power source, an air chamber and a reservoir 306 for containing an inhalable bicarbonate solution. The reservoir 306 may be removable from a receptacle 308 in the housing 304. The housing 304 further includes connectors and one-way valves (as described above for the delivery system 100) connecting the pump, the reservoir, the aerosolizing mechanism and the air chamber forming a conduit for passing the inhalable bicarbonate solution from reservoir 306 to the interior of the mask 302. The interior of the mask 302 may include a mouthpiece or a nasal cannula to direct inhalable bicarbonate into the user's mouth or nose.

Referring again to FIG. 5, in one embodiment, the housing 104 includes a cartridge receptacle 108, capable of accepting a quick load cartridge containing a pre-mixed amount of sodium bicarbonate solution. The quick load cartridge feature simplifies the experience for the user, whereby pre-mixed solutions at the precise effective dose can be purchased and inserted rapidly at time of use. This ensures the effective dose is always used and avoids the potential for users to prepare inconsistent doses. The quick load cartridge also promotes long-term purchases of supplies well beyond the initial purchase of the delivery system, thereby boosting the commercial potential of the design. Following loading of the cartridge with pre-mixed solution, the user wears the facemask and breathes the nebulized aerosol while sitting at rest.

According to embodiments, wherein the delivery system 100 is a nebulizer, the aerosolizing mechanism includes a nozzle, a porous membrane or a mesh through which the bicarbonate composition is pumped at pressure to form nebulized particles. According to other embodiments wherein the delivery system 100 is a vaporizer or an atomizer, the aerosolizing mechanism may include a heating element, an atomizer, or other means (e.g., sonication), for vaporizing or atomizing the inhalable bicarbonate composition.

According to an embodiment, the delivery system 100 may be integrated with sensors and/or wireless connectivity to sensors. The delivery system could monitor the patient's vital signs and acidity levels in real-time, adjusting the dosage and inhalation frequency accordingly. Additionally, the delivery system 100 may be able to sync with a smartphone app to track treatment progress and provide data to healthcare professionals.

According to some embodiments, the bicarbonate composition may be inhaled through a nasal inhaler configured to deliver sodium bicarbonate in a powdered or aerosolized form. This approach could offer a more targeted delivery method for specific acidosis conditions, such as respiratory acidosis.

According to some embodiments, there is an inhalation chamber that can administer precise doses of inhalable sodium bicarbonate. The chamber may be used in clinical settings or integrated into existing medical devices e.g., ventilators for patients with severe acidosis.

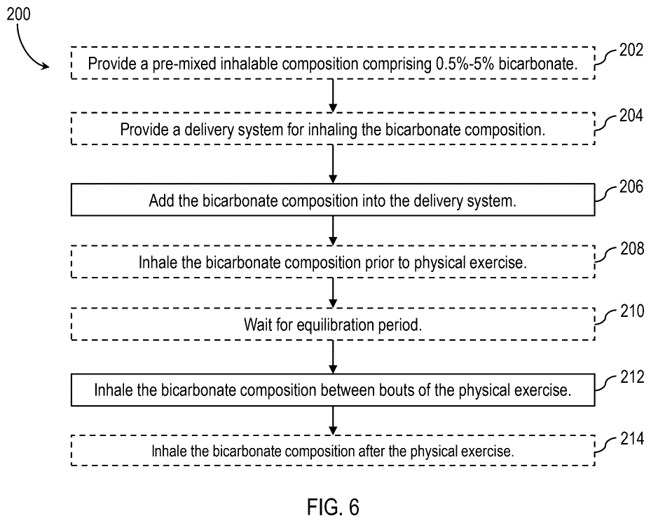

Referring to FIG. 6, shown therein is a flow chart of a method 200 for enhancing athletic performance, according to an embodiment. The method 200 comprises inhaling an effective amount of inhalable bicarbonate composition before engaging in athletic activity and/or between bouts of athletic activity. The method 200 may be used in combination with other ergogenic aids such as beta-alanine, caffeine, nitrates, creatine, etc., to enhance athletic performance.

According to some embodiments, at step 202 a pre-mixed inhalable composition comprising sodium bicarbonate may be provided in a vial, bottle, cartridge, or the like. The inhalable composition may be any one of the compositions described above having between 0.5% and 5% sodium bicarbonate by weight.

According to some embodiments, at step 204 a delivery system for inhaling the bicarbonate composition may be provided. According to an embodiment, the delivery system is the nebulizer shown in FIG. 5. According to other embodiments, the delivery system may be a vaporizer, atomizer or other delivery system suitable for aerosolizing the inhalable bicarbonate composition.

At 206, the inhalable bicarbonate composition is added into the delivery system e.g., the nebulizer of FIG. 5 as described above.

According to some embodiments, at step 208, the bicarbonate composition is inhaled, via the delivery system, while the user is at rest prior to exercise. For example, the bicarbonate composition may be inhaled at rate of ˜4-6 mL every 5-10 minutes for 30 minutes prior to commencing physical exercise.

According to some embodiments, at step 210 the user waits for an equilibration period before commencing physical exercise. Although the user can begin exercise immediately after step 208, an equilibration period of at least five minutes is recommended to ensure all of the bicarbonate has entered and equilibrated evenly in the blood prior to exercise.

At 212, the bicarbonate composition is inhaled, via the delivery system, between bouts of the physical exercise. Generally, the bicarbonate composition may be inhaled at regular or variable intervals during the physical exercise. For example, the bicarbonate solution may be inhaled for 60 seconds between bouts of the physical exercise, or inhaled at points during the physical exercise when the user feels the onset of fatigue or muscle cramps.

According to some embodiments, at step 214, the bicarbonate composition may be inhaled, via the delivery system, after completion of the physical exercise to prevent acidosis by normalizing blood pH and/or aid in post-exercise recovery.

While the method 200 is described in the context of enhancing athletic performance, the method 200 may be adapted for delaying the onset of acidosis or treating acidosis generally, regardless of the cause.

While the above description provides examples of one or more apparatus, methods, or compositions, it will be appreciated that other apparatus, methods, or compositions may be within the scope of the claims as interpreted by one of skill in the art.

1. A method of treating acidosis, comprising:
inhaling an effective amount of an inhalable bicarbonate composition via a delivery system.

2. The method of claim 1, wherein the inhalable bicarbonate composition is nebulized, atomized or vaporized by the delivery system.

3. The method of claim 1, wherein the delivery system is one of: a nebulizer, an atomizer and a vaporizer.

4. The method of claim 1, wherein the inhalable bicarbonate composition comprises sodium bicarbonate, potassium bicarbonate, another bicarbonate salt, or a mixture thereof.

5. The method of claim 1, wherein the effective amount is between 0.5% to 5%, and preferably about 3.5%, bicarbonate by weight, dissolved in water.

6. The method of claim 1, wherein the bicarbonate composition is inhaled at a rate of ˜4-6 mL every 5-10 minutes.

7. A method of enhancing athletic performance and recovery, comprising:
inhaling an effective amount of an inhalable bicarbonate composition, via a delivery system, prior to engaging in physical activity and/or between bouts of physical activity and/or following physical activity.

8. The method of claim 7, wherein the effective amount is between 0.5% to 5%, and preferably about 3.5% bicarbonate by weight, dissolved in water.

9. The method of claim 7, wherein the bicarbonate composition is inhaled at a rate of ˜4-6 mL every 5-10 minutes.

10. The method of claim 7, further comprising:
providing the delivery system, wherein the delivery system is one of: a nebulizer, an atomizer, a vaporizer and a nasal inhaler.

11. The method of claim 7, further comprising:
providing the inhalable bicarbonate composition as a pre-mixed solution in a vial, a bottle or a cartridge.

12. The method of claim 7, further comprising:
adding the inhalable bicarbonate composition to the delivery system.

13. The method of claim 7, further comprising:
waiting for an equilibration period after inhaling the bicarbonate composition prior to engaging in physical activity.

14. A delivery system, comprising:
a reservoir for containing a bicarbonate composition;
an aerosolizing mechanism, in fluidic connection with the reservoir, configured for generating inhalable bicarbonate;
a variable speed pump for forcing the bicarbonate composition through the aerosolizing mechanism to generate the inhalable bicarbonate;
a facemask; and
a conduit for directing the inhalable bicarbonate from the aerosolizing mechanism to the facemask.

15. The delivery system of claim 14, wherein the conduit comprises:
a chamber for mixing external air with the inhalable bicarbonate;
a one-way inspiratory valve configured to allow the external air into the chamber when the user inhales; and
a one-way expiratory valve configured to prevent back flow into the chamber when the user exhales.

16. The delivery system of claim 14, wherein the variable speed pump is manually adjustable by the user.

17. The delivery system of claim 14 further comprising:
a receptacle for receiving the reservoir, the reservoir having a top shaped to form fit or interference fit with the receptacle.

18. The delivery system of claim 14, wherein the bicarbonate composition is a pre-mixed solution of between 0.5% to 5%, and preferably about 3.5% sodium bicarbonate, by weight, dissolved in water.

19. An inhalable bicarbonate composition, comprising:
0.5% to 5%, and preferably about 3.5% sodium bicarbonate, potassium bicarbonate, another bicarbonate salt, or a mixture thereof, by weight;
water;
and flavoring.

20. The composition of claim 19, provided in one of: a single use disposable cartridge, a screw cap vial, a squeeze bottle, a dropper bottle and an inhalable strip.
